(19) United States
(12) Patent Application Publication
(10) Pub. No.: US 2021/0140858 A1
(43) Pub. Date: May 13, 2021

(54) INTESTINAL FLORA EXTRACTION VESSEL

(71) Applicant: OTSUKA PHARMACEUTICAL FACTORY, INC., Naruto-shi, Tokushima (JP)

(72) Inventors: Masaki NISHIOKA, Naka-gun (JP); Osamu TAKIGUCHI, Naka-gun (JP); Yasushi NAGATA, Naruto-shi (JP); Akihiro NABESHIMA, Naruto-shi (JP); Takashi FUJIMOTO, Naruto-shi (JP)

(73) Assignee: OTSUKA PHARMACEUTICAL FACTORY, INC., Naruto-shi, Tokushima (JP)

(21) Appl. No.: 16/488,190

(22) PCT Filed: Feb. 23, 2018
(86) PCT No.: PCT/JP2018/006646

§ 371 Date: Mar. 20, 2020

(30) Foreign Application Priority Data
Feb. 23, 2017 (JP) 2017-032664
Mar. 24, 2017 (JP) 2017-058792
Jul. 28, 2017 (JP) 2017-147201

(51) Int. Cl. G01N 1/40; C12M 1/00; C12M 3/00

(52) U.S. Cl. CPC G01N 1/4077; C12M 23/14; C12M 23/22; C12M 23/26; G01N 2001/4088; C12M 43/00; C12M 45/00; C12M 45/22; C12M 23/46

(57) ABSTRACT

The present invention provides an intestinal flora extraction vessel for extracting an intestinal flora from feces, the vessel including: a vessel main body with a first opening portion and a second opening portion formed therein, the vessel main body being configured so as to be deformable; an opening/closing mechanism configured to open/close the first opening portion of the vessel main body; a filter member accommodated in the vessel main body and partitioning the vessel main body into a first region located on the side of the first opening portion and a second region located on the side of the second opening portion, the filter member being configured to filter off the intestinal flora from the feces; and a communication member attached to the second opening portion, and configured to allow the second region to be in communication with the outside of the vessel main body.

### TECHNICAL FIELD

The present invention relates to an intestinal flora extraction vessel and an intestinal flora extraction method.

### BACKGROUND ART

In recent years, a medical treatment method called intestinal flora transplantation has attracted attention. In this method, digestive diseases such as ulcerative colitis, Crohn's disease, or the like are treated by transplanting intestinal flora contained in the feces of a person with a healthy balance of intestinal bacteria to a person whose balance of intestinal bacteria is disturbed. Also, treatment of various diseases such as obesity by adjusting the balance of intestinal bacteria is anticipated. Specifically, the feces of a healthy person and a solution such as physiological saline are mixed, and filtration is performed using a filter. Thus it is possible to obtain a solution containing the intestinal flora. Then, the solution containing the intestinal flora is injected into the large intestine of the subject person by an endoscope or the like.

Incidentally, in the above sort of method, there is a problem that the operation is uncomfortable because feces is handled. Therefore, for example, Patent Literature 1 discloses the following sort of method. First, after accommodating the feces in a first cup, an opening of the first cup is closed by a second cup to which a filter has been attached. At this time, the first and second cups are connected. Next, physiological saline is injected from a hole formed in the second cup, and the feces in the first cup is soaked with the physiological saline. Then, by shaking the first and second cups, the intestinal flora contained in the feces moves together with the physiological saline to the second cup through the filter. When a filtrate obtained in this way is collected from the second cup, the filtrate can be used for the above treatment.

### CITATION LIST

### Patent Literature

Patent Literature 1: U.S. Pat. No. 9,192,361

### SUMMARY OF INVENTION

### Technical Problem

However, in the above-described device, there is a problem that the operation such as connecting the cups and shaking the cups is complicated. There is also a problem that it is difficult to sufficiently filter off the intestinal flora from the feces by shaking. The present invention was made in order to address the above-described problems, and it is an object of the present invention to provide an intestinal flora extraction vessel and an intestinal flora extraction method such that it is possible to extract intestinal flora reliably and with a simple operation.

### Solution to Problem

The present invention provides an intestinal flora extraction vessel for extracting an intestinal flora from feces, the vessel including: a vessel main body with a first opening portion and a second opening portion formed therein, the vessel main body being configured so as to be deformable; an opening/closing mechanism configured to open/close the first opening portion of the vessel main body; a filter member accommodated in the vessel main body and partitioning the vessel main body into a first region located on the side of the first opening portion and a second region located on the side of the second opening portion, the filter member being configured to filter off the intestinal flora from the feces; and a communication member attached to the second opening portion, and configured to allow the second region to be in communication with the outside of the vessel main body. The filter member is formed in a bag shape that has an internal space open to the side of the first opening portion.

According to this configuration, because the filter member is accommodated within the deformable vessel main body, by applying an external force to the vessel main body after the feces is accommodated into the vessel main body from the first opening and the solution is injected, it is possible to filter off the intestinal flora together with the solution from the feces using the filter member. As a result, it is possible to obtain a filtrate containing the intestinal flora in the second region. This allows the operator to extract the filtrate without directly touching the feces. Also, although there is no direct touching of the feces, for example, because it is possible to press the vessel by hand to squeeze the feces, or shake the vessel, it is possible to reliably exert a force on the feces, and so the intestinal flora can be reliably extracted from the feces. Therefore, according to this example embodiment, it is possible to suppress discomfort of the operation, and to reliably extract the filtrate containing the intestinal flora.

In the above intestinal flora extraction vessel, the filter member can be formed in a bag shape that has an internal space open to the side of the first opening portion.

According to this configuration, the feces can be accommodated within the filter member, and an external force can be applied to the feces using the entire filter member to squeeze the feces. Alternatively, the feces can be rubbed or broken within the filter member by shaking the vessel main body. Therefore, filtration of the feces can be performed efficiently because the area of the filter member for filtering the solution is increased.

In the above intestinal flora extraction vessel, the opening/closing mechanism can be configured to close the first opening portion in a sealed state.

According to this configuration, the feces can be sealed within the vessel main body, so the odor of the feces can be blocked.

In the above intestinal flora extraction vessel, a gap can be formed between an end portion located on the side of the second opening portion of the filter member and the second opening portion.

According to this configuration, a gap is formed between the end portion of the filter member and the communication member, so it is possible to easily visually recognize that the filtrate has accumulated in the second region. Also, it is possible to prevent the feces or the filter member from being caught in a through-hole of the communication member, and therefore the filtrate can smoothly flow into the through-hole.

In each intestinal flora extraction vessel above, a gap can be formed between an end portion located on the side of the first opening portion of the filter member and the first opening portion.

According to this configuration, a gap is formed between the end portion of the filter member and the first opening portion, so it is possible to provide a predetermined distance between the feces accommodated in the filter member and the first opening portion. Thus it is possible to prevent feces from contacting the first opening portion, and therefore it is possible to reliably close the first opening portion.

In each intestinal flora extraction vessel above, the communication member can be configured such that a syringe, a bag, or a bottle can be connected to the communication member. Thus, it is possible to easily extract the filtrate from the vessel main body.

In the above intestinal flora extraction vessel, it is possible to further provide a fixing means for fixing the communication member to a syringe, a bag, or a bottle. Thus, for example, while the communication member and the syringe, the bag, or the bottle remain fixed, after injecting the solution from the syringe or the like into the vessel main body, it is possible to discharge the solution containing the intestinal flora into the syringe or the like. Accordingly, it is possible to continuously perform the operation of extracting the solution containing the intestinal flora, without removing the syringe or the like.

In each intestinal flora extraction vessel above, at least a part of the vessel main body can be colored. Thus, it is possible for the feces to be invisible from the outside, so it is possible to suppress discomfort of the operation. As the coloring method, it is possible to adopt a method of mixing a coloring agent with a film used to form the vessel main body, or to apply a coloring ink to at least one face of the vessel main body.

In each intestinal flora extraction vessel above, at least part of the filter member can be colored. Thus, it is possible for the feces to be invisible from the outside, so it is possible to suppress discomfort of the operation. As the coloring method, it is possible to mix a coloring agent with a material used to form the filter.

In each intestinal flora extraction vessel above, all of the vessel main body can be transparent. Thus, it is possible for the feces to be easily viewed from the outside, so feces filtration and intestinal flora extraction can be performed reliably.

In each intestinal flora extraction vessel above, all of the filter member can be transparent. Thus, it is possible for the feces to be easily viewed from the outside, so feces filtration and intestinal flora extraction can be performed reliably.

In each intestinal flora extraction vessel above, it is possible to further provide a support member attached to the vessel main body, and capable of attaching the vessel main body to a rim of a toilet in a state with the first opening portion opened upward.

Thus, the feces discharged from a person can be directly accommodated in the vessel main body fixed to the toilet by the support member. Thus, an uncomfortable operation of accommodating the feces in the vessel main body can be omitted.

In each intestinal flora extraction vessel above, it is possible to further provide an outer vessel configured to cover the intestinal flora extraction vessel. Thus, it is possible to apply external force to the vessel main body through the external vessel, rather than applying external force directly to the vessel main body, and therefore an uncomfortable operation such as squeezing the feces can be reduced.

In each outer vessel above, at least a part of the outer vessel can be non-transparent or colored. Thus, even if the vessel main body or the filter member is transparent, the feces is not viewed directly, so discomfort of the operation can be suppressed.

In each intestinal flora extraction vessel above, it is possible to adopt a configuration in which the vessel main body is formed in a rectangular shape that has a pair of opposing end portions, and a side portion between the end portions, the first opening portion being provided in either one of the end portions, and the second opening portion being provided in the side portion.

According to this configuration, for example, when the vessel main body is disposed in the toilet such that the first opening portion faces upward, the second opening portion is disposed in the side portion of the vessel main body, so the communication member to be attached to the second opening portion does not face downward, but faces sideways. Therefore, the communication member can be prevented from coming into contact with water or sewage accumulated in the toilet. Note that the term “side portion” includes not only the side face of the vessel main body but also a corner portion that is an intersecting portion where the end portion and the side portion intersect.

An intestinal flora extraction method according to the present invention includes: a step of preparing a deformable vessel main body partitioned into a first region and a second region by a bag-shaped filter member; a step of accommodating feces from the side of the first region into the filter member of the vessel main body, and closing the vessel main body; a step of, before closing the vessel main body or after closing the vessel main body, accommodating a solution into the filter member; a step of applying external force to the vessel main body, and filtering off intestinal flora contained in the feces, together with the solution, from the filter member to the second region; and a step of removing the solution containing the intestinal flora, accommodated in the second region, to the outside of the vessel main body.

In the above intestinal flora extraction method, a method can be adopted such that in the step of accommodating the solution, a syringe, a bag, or a bottle is connected to the vessel main body, and the solution is injected from the syringe, the bag, or the bottle into the vessel main body, and in the step of removing the solution containing the intestinal flora to the outside of the vessel main body, the solution containing the intestinal flora is discharged into the syringe, the bag, or the bottle, which remains connected to the vessel main body.

In the above intestinal flora extraction method, the solution can be selected from at least one of physiological saline, Ringer's solution, Ringer's lactate solution, and a solution to which a component with an excellent ability to support the growth of intestinal flora has been added.

In the above intestinal flora extraction method, the component with an excellent ability to support the growth of intestinal flora can be at least one of an amino acid, a sugar, a mineral, a vitamin, and lactic acid bacteria, or the component can be a yogurt containing at least one of these.

In the above intestinal flora extraction method, it is possible to further provide a step of attaching the prepared vessel main body to a toilet such that the first region side opens, and to adopt a method such that in the step of accommodating the feces into the filter member, the feces discharged by a subject person sitting on the toilet is directly accommodated into the filter member.

In the above intestinal flora extraction method, it is possible to further provide a step of covering the vessel main body with an outer vessel, and to perform the application of external force to the vessel main body from the outside of the outer vessel.

In the above intestinal flora extraction method, at least a part of the outer vessel can be non-transparent or colored.

### Advantageous Effects of the Invention

According to the present invention, it is possible to extract intestinal flora reliably and with a simple operation.

### BRIEF DESCRIPTION OF DRAWINGS

FIG. 1 is a front view of an intestinal flora extraction bag according to an example embodiment of the present invention;

FIG. 2 is a perspective view of a communication member;

FIG. 3 is a front view of a filter member;

FIG. 4 shows a method of using the intestinal flora extraction bag in FIG. 1;

FIG. 5 shows a method of using the intestinal flora extraction bag in FIG. 1;

FIG. 6 shows a method of using the intestinal flora extraction bag in FIG. 1;

FIG. 7 shows a method of using the intestinal flora extraction bag in FIG. 1;

FIG. 8 is a perspective view showing another example of the intestinal flora extraction bag in FIG. 1;

FIG. 9 is a perspective view showing a usage example of the intestinal flora extraction bag in FIG. 8;

FIG. 10 is a cross-sectional view of FIG. 9;

FIG. 11 is a perspective view showing another example of the intestinal flora extraction bag in FIG. 1;

FIG. 12 is a side view of the intestinal flora extraction bag in FIG. 11;

FIG. 13 is a side view showing another example of the intestinal flora extraction bag in FIG. 1;

FIG. 14 is a side view showing another example of the intestinal flora extraction bag in FIG. 1;

FIG. 15 is a side view showing another example of the intestinal flora extraction bag in FIG. 1; and

FIG. 16 is a side view showing another example of the intestinal flora extraction bag in FIG. 1.

### DESCRIPTION OF EMBODIMENTS

Following is a description of example embodiments of an intestinal flora extraction vessel and an intestinal flora extraction method according to the present invention, with reference to the attached drawings. Here, an intestinal bacterial flora extraction bag will be described as one example embodiment of the intestinal flora extraction vessel. FIG. 1 is a front view of the intestinal flora extraction bag according to this example embodiment, FIG. 2 is a front view of a communication member, and FIG. 3 is a front view of a filter member. In the following description, for convenience of explanation, the vertical direction in FIG. 1 is referred to as the “vertical direction”, and the horizontal direction in FIG. 1 is referred to as the “lateral direction”, the “width direction”, or the “horizontal direction”, for example. However, the orientation of the intestinal flora extraction bag is not limited by this description.

### 1. Intestinal Flora Extraction Bag

The intestinal bacterial flora extraction bag according to this example embodiment is provided with a bag main body 1 formed in a rectangular shape, and a filter member 2 to be accommodated in the bag main body 1. Each member will be described below.

### 1-1. Bag Main Body

As shown in FIG. 1, the bag main body 1 is formed by overlaying two deformable rectangular sheet members 101 and 102 made of resin, and side portions 11 on both sides in the width direction are fixed by heat sealing so as to form an internal space. Also, at an upper end portion of the bag main body 1, a first opening portion 14 is formed across the entire width in the horizontal direction. As shown in the enlarged view in FIG. 1, on the inner faces of the respective sheet members 101 and 102, a recessed section 12 and a protruding section 13 are formed in the width direction so as to follow along the first opening portion 14. The first opening portion 14 is configured to be capable of opening/closing by detachably fitting the recessed section 12 and the protruding section 13 together. Furthermore, when the recessed section 12 and the protruding section 13 have been fitted together, the first opening portion 14 is closed in a sealed state. Note that the recessed section and the protruding section 13 correspond to the opening/closing mechanism of the present invention. Also note that it is preferable that the opening/closing mechanism of the present invention has a structure in which backflow is unlikely, such that feces and material extracted from the feces do not leak. Also, as the opening/closing mechanism, instead of providing recessed and protruding fittings within the bag, it is possible, for example, to close the opening portion in a sealed state by sandwiching the outer faces of both sheet members 101 and 102 with a clip.

On the other hand, heat sealing is performed at a lower end portion of the bag main body 1, except near the center in the horizontal direction, and the center portion where heat sealing is not performed is used to configure a second opening portion 15. Furthermore, the two sheet members 101 and 102 are fixed in the location where heat sealing is performed. Also, a communication member 3 made of resin is fitted into the second opening portion 15, and the surfaces of the communication member 3 and the sheet members 101 and 102 are fixed by heat sealing. Thus, the second opening portion 15 is closed in a sealed state.

As shown in FIG. 2, the communication member 3 has a cross-sectional shape in which arcs facing in opposite directions from each other are combined, and has two curved face portions 31 respectively facing the sheet members 101 and 102, and an upper face 32 and a lower face 33 that point in the vertical direction. Also, a through-hole 34 is formed passing through the upper face and the lower face. The through-hole 34 allows the internal space of the bag main body 1 to be in communication with the outside.

A connector 4 for connecting a syringe 5 is attached to the communication member 3 in order to inject physiological saline, as described later. Although this connector 4 can be implemented in various modes, a configuration can be adopted in which, for example, a check valve is incorporated, and when the syringe 5 is not connected, the check valve prevents the outflow of liquid or the like from the communication member 3 side, and when the syringe 5 is connected, the check valve opens, causing the syringe 5 and the through-hole 34 of the communication member 3 to be in communication with each other.

The materials forming the sheet members 101 and 102 of the bag main body 1 are, of course, deformable and waterproof, but are not particularly limited. For example, polypropylene (PP), polyethylene (PE), or the like can be used. Also, the thickness of the sheet members 101 and 102 is set to, for example, 50 to 200 μm in order to obtain appropriate strength, and as described later, in order to obtain flexibility such that squeezing of the feces is not hindered.

### 1-2. Filter Member

Next is a description of the filter member 2. As shown in FIG. 3, in the filter member 2, heat sealing is performed on both sides and at the lower end portion of two sheet members 201 and 202, thus forming the filter member 2 in a bag shape with an internal space that opens at the upper end portion. The peripheral edge of the upper end portion is fixed to the inner wall face of the bag main body 1 by heat sealing. Thus, the filter member 2 is attached so as to open on the side of the upper end portion of the bag main body 1, and as shown in FIG. 1, the filter member 2 partitions the internal space of the bag main body 1 into a first region 10A located on the side of the upper end portion and a second region 10B located on the side of the lower end portion.

Also, as shown in FIG. 1, a gap S1 is formed between the upper end opening of the filter member 2 and the upper end portion of the bag main body 1, that is, the first opening portion 14. Also, a gap S2 is formed between the lower end portion of the filter member 2 and the lower end portion of the bag main body 1, that is, the second opening portion 15. The size of the filter member 2 is not particularly limited, but as described later, because it is necessary to accommodate the feces and filter out the feces from small pores while squeezing the feces, the filter member 2 preferably is large enough to be capable of accommodating all of the feces. Therefore, for example, a length L in the vertical direction of the filter member 2 is preferably 50 mm or more, more preferably 100 mm or more, and particularly preferably 200 mm or more. Also, the length of the filter member 2 in the horizontal direction is, for example, preferably 50 mm or more, more preferably 100 mm or more, and particularly preferably 200 mm or more.

The filter member 2 is formed of a material in which pores are formed, such as non-woven fabric, filter paper, cloth, hollow fiber, sponge, porous metal, polyamide mesh, deformable porous metal such as wire mesh, or the like. The pores need to be large enough for the intestinal bacteria contained in the feces to pass through the material of the filter member. Also, when connecting a syringe, a bag, or a bottle to the communication member and extracting the filtrate, there is a risk that the connecting portion will become clogged by undigested material or the like in the feces, so it is preferable that the pores have a size that does not allow such material to pass through the material of the filter member.

Specifically, because the intestinal bacteria have an outer diameter of about 0.1 to 20 μm, for example, the lower limit of the pore diameter is preferably 0.1 μm or more, and depending on the type of intestinal bacteria intended for extraction, the lower limit of the pore diameter can be set to 1 μm or more or 5 μm or more. This allows the intestinal bacteria to pass through the material of the filter member.

Also, the connecting portion of the communicating member, or the syringe, the bag, or the bottle, has an inner diameter of about 1 to 10 mm. If the pore diameter is made larger than this, undigested material or the like in the feces of a size such that the connecting portion becomes clogged also passes through the filter member 2, and there is a risk of hindering extraction of the filtrate. From this viewpoint, the pore diameter is preferably 10 mm or less, more preferably 3 mm or less, and still more preferably 1 mm or less. Thus it is possible to effectively extract the filtrate. Also, as described later, the thickness of the filter member 2 is preferably 10 μm to 10 mm, for example, in order to have flexibility such that squeezing the feces is possible, and a strength so as to not break when squeezing is performed.

A plurality of filter members 2 can also be used. For example, it is possible to use a plurality of the filter members 2 in a stacked manner, or to dispose the plurality of filter members 2 with a gap between them, and obtain intestinal flora that has passed through the plurality of filter members 2 in the second region 10B. In this case, the pore diameter of the filter member disposed on the side of the first opening portion 14 can be increased, and the pore diameter of the filter member disposed on the side of the second opening portion 15 can be reduced. Thus, while preventing clogging of the pores, it is possible to prevent the undigested material in the feces, which may hinder administration of the extraction solution, from flowing into the second region 10B.

### 2. Method of Using Intestinal Flora Extraction Bag

Next, a method of using the intestinal bacterial flora extraction bag configured as described above will be described. First, as shown in FIG. 4, the first opening portion 14 is opened, and a feces 90 to be used for transplantation is accommodated in the bag main body 1. For example, 10 to 500 g of feces can be accommodated.

At this time, the feces is accommodated within the filter member 2 within the bag main body 1. As described above, because the filter member 2 is formed in a bag shape, feces is held within the filter member 2. Next, the recessed section 12 and the protruding section 13 of the first opening portion 14 are fitted together to seal the first opening portion 14. Then, as shown in FIG. 5, the syringe 5 is connected to the connector 4, which has been attached to the communication member 3, and physiological saline is injected into the bag main body 1. For example, 10 to 500 mL of physiological saline can be injected, depending on the amount of feces that is accommodated.

The physiological saline penetrates from the second region 10B of the bag main body 1 through the filter member 2 into the first region 10A, and the feces is immersed in the physiological saline. Subsequently, after the syringe 5 is removed from the connector 4, as shown in FIG. 6, the operator manually grips a position corresponding to the filter member 2 on the outer face of the bag main body 1 and squeezes the feces. Thus, the intestinal flora contained in the feces mixes with the physiological saline and flows out together with the physiological saline through the filter member 2 to the second region 10B of the bag main body 1. That is, the intestinal flora is filtered off together with the physiological saline from the feces by the filter member 2. Hereinafter, the physiological saline containing the intestinal flora that has flowed out to the second region 10B will be referred to as the filtrate. When a predetermined amount of the filtrate has accumulated in the second area 10B due to squeezing the bag main body 1 in this way, as shown in FIG. 7, the syringe 5 is connected to the connector 4, and the filtrate accumulated in the second region 10B is sucked out.

The filtrate extracted as described above is injected into the large intestine of the subject person to be treated with an endoscope or the like. Note that it is preferable to anaerobically perform the operation for extraction of intestinal flora described above. In order to do so, for example, it is possible to quickly perform a filtration operation after the feces is accommodated in the bag main body 1, or to enclose nitrogen in the bag main body 1, in an outer bag described later, or the like. Also, after extracting the filtrate using the bag main body 1, a further process such as filtration can be performed on the filtrate.

### 3. Features

As described above, according to this example embodiment, the following effects can be obtained.

(1) In extracting the filtrate containing the intestinal flora from the feces, the feces is accommodated in the filter member 2 within the bag main body 1, and after physiological saline is injected, the bag main body 1 is squeezed, and the intestinal flora is filtered off together with the physiological saline from the feces, thus obtaining the filtrate. This allows the operator to extract the filtrate without directly touching the feces. Also, because the feces is sealed within the bag main body 1, the odor of the feces can be blocked. Also, because the filtration can be performed by only manually squeezing the bag main body 1, the operation is simple. Furthermore, although there is no direct touching of the feces, the feces can be manually squeezed, so the intestinal flora can be reliably extracted from the feces. Therefore, according to this example embodiment, it is possible to suppress the discomfort of the operation, and reliably extract the filtrate containing the intestinal flora.
(2) A gap is formed between the lower end portion of the filter member 2 and the communication member 3, so it is possible to easily visually recognize that the filtrate has accumulated in the second region 10B. Also, the filter member 2 can be prevented from being caught in the through-hole 34 of the communication member 3, and therefore the filtrate can smoothly flow into the through-hole 34.
(3) A gap is formed between the upper end portion of the filter member 2 and the first opening portion 14, so it is possible to provide a predetermined distance between the feces accommodated in the filter member 2 and the first opening portion 14. Thus, it is possible to prevent feces from contacting the first opening portion 14, and therefore it is possible to reliably close the first opening portion 14.

### 4. Modified Examples

Although one example embodiment of the present invention is described above, the present invention is not limited by the above example embodiment, and various modifications are possible without departing from the gist of the invention. Also, the modified examples described below can be combined as appropriate.

4-1

The color of the bag main body 1 and the filter member 2, other than being transparent, is not particularly limited, but for example, by coloring them it is possible for the feces to be invisible from the outside. If the bag main body 1 and the filter member 2 are transparent, feces filtration and intestinal flora extraction can be performed reliably because the feces can be easily viewed from the outside. On the other hand, if the bag main body 1 and the filter member 2 are colored, the operator can perform the operation without directly looking at the feces, so discomfort can be reduced.

4-2

In order for the bag main body 1 and the filter member 2 to be non-transparent or colored, it is possible to use a known method, such as adopting a material such as aluminum foil for the bag main body 1, or coating the bag main body 1 and the filter member 2 with ink, or mixing with a coloring agent. Also, non-transparency or coloring may be implemented only in a part of the bag main body 1 rather than the entire bag main body 1. For example, a configuration may be adopted in which the portion corresponding to the second region 10B is transparent and other portions are non-transparent or colored, such that the filtrate can be confirmed.

4-3

When the bag main body 1 and the filter member 2 are transparent, they can be covered with a non-transparent or colored outer vessel. That is, after the feces and the solution are placed into the bag main body 1, the bag main body 1 can be accommodated in the outer vessel to be squeezed. Thus, the operator can perform the operation without looking directly at the feces, so discomfort can be reduced. Note that the outer vessel, other than being formed in a bag shape so as to be able to accommodate the bag main body 1, can also be formed so as to simply cover the periphery of the bag main body 1.

4-4

The outer vessel, like the bag main body 1, for example, can be formed with a deformable material formed in a bag shape. Also, in order for the outer vessel to be non-transparent or colored, it is possible to use a known method, such as adopting a material such as aluminum foil for the outer vessel, or coating the outer vessel with ink, or mixing with a coloring agent. With this outer vessel as well, as in the case of the bag main body 1, it is possible to adopt a configuration in which one part is transparent and another part is colored.

4-5

Furthermore, a deodorizing function or an odor-emitting function may be provided in the bag main body 1 and the filter member 2, or the outer vessel. In order to provide a deodorizing function, an adsorbent material that has a deodorizing function can be mixed into a film forming the bag main body 1 and the filter member 2, or the outer vessel. In order to provide an odor-emitting function, a fragrance or the like can be provided inside of the outer vessel.

4-6

Furthermore, gas barrier properties may be provided to the bag main body 1 or the outer vessel. In order to provide the gas barrier properties, aluminum foil or a commonly known gas barrier film can be used for the bag main body 1 or the outer vessel.

4-7

In the above example embodiment, the syringe 5 is connected to the communication member 3 and then the physiological saline is injected. However, a configuration may also be adopted in which, while maintaining the connection of the syringe 5, which has been used to inject the physiological saline, the bag main body 1 is squeezed, and thus the filtrate is extracted using the connected syringe 5. Also, a configuration may be adopted in which, instead of injecting the physiological saline using the syringe 5, for example, the physiological saline is poured together with the feces from the first opening portion 14.

4-8

The configuration of the communication member 3 is not particularly limited, but a configuration may also be adopted in which, instead of the syringe 5, for example, a bag in which physiological saline has been accommodated is connected to the communication member 3, and then the physiological saline is injected from this bag. Therefore, a configuration may be adopted in which a connector that is configured such that a syringe, a bag, or a bottle for injecting physiological saline, or for extracting the filtrate, can be connected thereto, and when these are not connected, the through-hole 34 of the communication member 3 is closed, can be connected to the communication member 3. Alternatively, a configuration may be adopted in which the communication member 3 has such a function.

Also, the main body bag 1 can be fixed with the syringe 5, a bag, or a bottle (hereinafter, these three are combined and referred to as “the syringe or the like”) in a connected state. For example, in this sort of fixing it is possible to use a connecting member (fixing means) that has needles at both ends, such as double-ended needles, and that has communication holes inside these needles. That is, after the communication member 3 (or the connector 4) and the syringe or the like are connected by such a connection member, the connection member is fixed to the communication member 3 and the syringe or the like. Thus, because the bag main body 1 and the syringe or the like are fixed, it is possible to omit the work of removing the bag main body 1 and the syringe or the like when injecting the physiological saline and when extracting the filtrate, and as a result it is possible to continuously perform the intestinal flora extraction operation. Note that the method of fixing the connection member is not particularly limited. For example, the shape of the connection member can be such that the connection member can be fixed to the syringe or the like, or the connection member can be connected to the bag main body 1 or the like by another fixing member.

4-9

It is sufficient that at least part of the filter member 2 is connected to the bag main body, but a configuration may also be adopted in which, for example, a side edge of the filter member 2 is fixed by heat sealing together with both side portions 11 of the bag main body 1. This configuration facilitates manufacturing. Also, the shape of the filter member 2 is not particularly limited, and a configuration other than a bag shape in which an internal space can accommodate feces, as in the above example embodiment, is also possible. For example, the filter member 2 may be a sheet-like member that simply partitions the bag main body 1 into the two regions 10A and 10B. With this configuration, for example, it is possible to squeeze feces together with the solution in the first region 10A, and filter this mixture to obtain a filtrate in the second region 10B.

Also, the fixing position of the filter member 2 and the bag main body 1 is not particularly limited, and may be a position other than the upper end portion of the filter member 2, and it is sufficient that the bag main body 1 partitions the bag main body 1 into the two regions 10A and 10B. Also, the bag main body 1 and the filter member 2 can also be formed by a method other than heat sealing. The method of forming the bag main body 1 and the filter member 2 is not particularly limited, and for example, ultrasonic fusion, a tape, an adhesive agent, or the like may be used.

4-10

In the above example embodiment, the first opening portion 14 is closed with a recessed/protruding fitting using the recessed section 12 and the protruding section 13, but another configuration can be adopted, and this configuration is not particularly limited as long as it has a mechanism such that opening/closing is possible. For example, various configurations are applicable, such as a clip or a slidable chuck. Also, it is not necessary that the first opening portion 14 is able to be tightly closed in a fluid-tight or air-tight manner, and it is sufficient that the first opening portion 14 is provided with a mechanism such that leakage of the feces or the solution to the outside can be prevented.

4-11

The intestinal flora extraction bag configured as described above can be placed in a toilet to collect feces directly from the human body. For example, as shown in FIG. 8, sheet-like support members 7 are attached to opposing outer faces of the bag main body. The support members 7 each can be formed of deformable paper or plastic so as to conform to the shape of a toilet, in particular the shape of the rim of a toilet bowl, and it is also possible to extend the upper end portion of the bag main body 1 to form the support members 7. Then, as shown in FIGS. 9 and 10, this intestinal flora extraction bag is attached to a toilet 8. At this time, the support members 7 are hooked on the rim of a toilet bowl 81 in a state with the first opening portion 14 opened upward. Subsequently, a toilet seat 82 is lowered, and the support members 7 are fixed sandwiched between the toilet bowl 81 and the toilet seat 82. In this state, if the feces collection subject has a bowel movement, the feces enters the filter member 2 from the first opening portion 14, so the feces can be collected directly. That is, it is possible to omit the operation by the operator for accommodating the feces in the bag main body 1. Also, although not shown, the intestinal flora extraction bag is placed upward in FIG. 10 so that when the feces collection subject is seated on the toilet seat, the collection subject and the bag main body 1 are allowed to be in close contact. Note that the attachment position of the intestinal flora extraction bag to the toilet 8 can be changed as appropriate depending on the shape of the toilet 8 and the method of collecting feces. For example, from the positions shown in FIGS. 9 and 10, the attachment position can be arbitrarily moved vertically or horizontally.

The configuration of the support members 7 is not particularly limited as long as the bag main body 1 can be attached to the toilet with the first opening portion 14 opened upward. Therefore, the shape and the material of the support members 7 are not particularly limited.

The support members 7 can also be fixed to the toilet 8 by a method other than the method described above. For example, after the support members 7 are passed over the toilet seat 82, the support members 7 may be fixed sandwiched between the toilet bowl 81 and the toilet seat 82. In this case, the support members 7 are spread under the buttocks of the collection subject. As a result, the bag main body 1 can be disposed upward, so the distance between water or sewage accumulated in the toilet 8 and the bag main body 1 increases, and the bag main body 1 can be prevented from contacting sewage or the like. Also, because the distance between the collection subject and the first opening portion 14 of the bag main body 1 decreases, it becomes easier to collect feces without leakage.

Also, a configuration may be adopted in which the bag main body 1 is fixed by attaching an adhesive means (such as a double-sided tape or an adhesive) to portions of the support members 7 and adhering the toilet seat 82 to the portions to which the adhesive means is attached. By adopting such a configuration, the support members 7 can easily be attached to the toilet seat 82, and displacement can be prevented.

4-12

In each description above, the first opening portion 14 is provided in the upper end portion of the bag main body 1, and the second opening portion 15 is provided in the lower end portion of the bag main body 1, but this is not a limitation of the present invention. These positions can be determined as appropriate according to the application. That is, the opening portions 14 and 15 do not necessarily have to be opposed to each other. For example, as shown in FIGS. 11 and 12, the second opening portion 15 can be provided in a side portion of the bag main body 1. That is, the second opening portion 15 can be disposed so as to not face downward but to face sideways. With this configuration, for example, as shown in FIG. 10, when the intestinal flora extraction bag is disposed on the toilet 8, the second opening portion 15 is disposed on the side face of the bag main body 1, and therefore, the communication member 4 attached to the second opening portion 15 can be prevented from contacting water or sewage accumulated in the toilet 8.

Also, a configuration can be adopted in which, as shown in FIG. 13, an inclined portion is provided at a corner of the lower end portion of the bag main body 1, that is, at a position where the side portion and the lower end portion of the bag main body 1 intersect, and the second opening portion 15 is formed in this inclined portion. According to this configuration as well, the communication member 4 provided in the second opening portion 15 does not face downward, so it is possible to prevent the communication member 4 from contacting water or sewage accumulated in the toilet 8.

4-13

In the example shown in FIG. 12, the filter member 2 is formed such that the lower end portion of the filter member 2 is located above the second opening portion 15, but the shape of the filter member 2 is not limited to this. For example, as shown in FIG. 14, the filter member 2 can be formed such that the filter member 2 is extended to the vicinity of the lower end portion of the bag main body 1, and the side portion of the filter member 2 is separated from the side portion of the bag main body 1 where the second opening portion 15 is provided. With this configuration, a gap is formed between the filter member 2 and the communication member 3, so it is possible to easily visually recognize that the filtrate has accumulated in the second region 10B. Also, the filter member 2 can be prevented from being caught in the communication member 3.

Alternatively, it is possible to adopt a configuration in which, as shown in FIG. 15, the filter member 2 is formed so as to bring the side portion of the filter member 2 close to the side portion of the bag main body 1 where the second opening portion 15 is provided, but a recessed portion 25 is only formed in the portion opposing the second opening portion 15. Therefore, in the internal space of the bag main body 1, the portion where this recessed part 25 is formed serves as the second region 10B. Even with such a configuration, as described above, it is possible to easily confirm the filtrate, and the filter member 2 can be prevented from being caught in the communication member 3.

4-14

The number of second opening portions 15 is not limited to one, and it is also possible to provide two or more of the second opening portions 15. Furthermore, a communication member 4 can be attached to each second opening portion 15 respectively. For example, as shown in FIG. 16, when two of the second opening portions 15 are provided and a communication member 4 is provided in each second opening portion 15, it is possible to use one communication member 4 for injecting the solution into the bag main body 1, and to use the other communication member 4 to extract the filtrate from the bag main body 1. Alternatively, if the filtrate is discharged using two of the communication members 4, the discharge time can be shortened.

4-15

In the above example embodiment, the bag main body 1 is disclosed as one aspect of the vessel main body forming the intestinal flora extraction vessel according to the present invention, but the shape and structure thereof are not particularly limited, and it is sufficient to adopt a configuration in which the filter member 2 can be accommodated. Therefore, it is not necessary to overlap two sheet members, as long as the vessel main body is formed in a bag shape. Alternatively, rather than forming the vessel main body in a bag shape, the vessel main body may be formed as a bottle or the like with a hard material. In this case, instead of extracting the intestinal flora by squeezing, it is possible to establish a sealed state after placing the feces and the physiological saline and the like in the bottle, and then extract the intestinal flora by shaking the bottle.

4-16

As the solution to be mixed with the feces, other than the above-mentioned physiological saline, it is possible to use Ringer's solution, Ringer's lactate solution, or a solution to which a component with an excellent ability to support the growth of intestinal flora has been added. Note that, in the case of a solution containing a component that can be sterilized, such as physiological saline or Ringer's solution, it is preferable to perform sterilization.

Also, as the above-mentioned component with an excellent ability to support the growth of intestinal flora, it is possible to use an amino acid, a sugar, a mineral, a vitamin, lactic acid bacteria, or the like, or a yogurt containing these may be used.

### LIST OF REFERENCE NUMERALS

1 Bag main body (vessel main body)
12 Recessed section (opening/closing mechanism)
13 Protruding section (opening/closing mechanism)
2 Filter member
3 Communication member
5 Syringe

1. An intestinal flora extraction vessel for extracting an intestinal flora from feces, the vessel comprising:
a vessel main body with a first opening portion and at least one second opening portion formed therein, the vessel main body being configured so as to be deformable;
an opening/closing mechanism configured to open/close the first opening portion of the vessel main body;
a filter member accommodated in the vessel main body and partitioning the vessel main body into a first region located on the side of the first opening portion and a second region located on the side of the second opening portion, the filter member being configured to filter off the intestinal flora from the feces; and
a communication member attached to the second opening portion, and configured to allow the second region to be in communication with the outside of the vessel main body.

2. The intestinal flora extraction vessel according to claim 1, wherein the filter member is formed in a bag shape having an internal space open to the side of the first opening portion.

3. The intestinal flora extraction vessel according to claim 1, wherein the opening/closing mechanism is configured to close the first opening portion in a sealed state.

4. The intestinal flora extraction vessel according to claim 1, wherein a gap is formed between an end portion located on the side of the second opening portion of the filter member and the second opening portion.

5. The intestinal flora extraction vessel according to claim 1, wherein a gap is formed between an end portion located on the side of the first opening portion of the filter member and the first opening portion.

6. The intestinal flora extraction vessel according to claim 1, wherein the communication member is configured such that a syringe, a bag, or a bottle can be connected to the communication member.

7. The intestinal flora extraction vessel according to claim 6, further comprising: a fixing means for fixing the communication member to a syringe, a bag, or a bottle.

8. The intestinal flora extraction vessel according to claim 1, wherein at least a part of the vessel main body is colored.

9. The intestinal flora extraction vessel according to claim 1, wherein at least a part of the filter member is colored.

10. The intestinal flora extraction vessel according to claim 1, wherein all of the vessel main body is transparent.

11. The intestinal flora extraction vessel according to claim 1, wherein all of the filter member is transparent.

12. The intestinal flora extraction vessel according to claim 1, further comprising:
a support member attached to the vessel main body, and capable of attaching the vessel main body to a rim of a toilet in a state with the first opening portion opened upward.

13. The intestinal flora extraction vessel according to claim 1, further comprising:
an outer bag configured to cover the vessel main body.

14. The intestinal flora extraction vessel according to claim 13,
wherein at least a part of the outer bag is non-transparent or colored.

15. The intestinal flora extraction vessel according to claim 1, wherein
the vessel main body is formed in a rectangular shape having a pair of opposing end portions, and a side portion between the end portions,
the first opening portion being provided in either one of the end portions, and
the second opening portion being provided in the side portion.

16. An intestinal flora extraction method comprising:
a step of preparing a deformable vessel main body partitioned into a first region and a second region by a filter member;
a step of accommodating feces from the side of the first region into the filter member of the vessel main body, and closing the vessel main body;
a step of, before closing the vessel main body or after closing the vessel main body, accommodating a solution into the filter member;
a step of applying external force to the vessel main body, and filtering off intestinal flora contained in the feces, together with the solution, from the filter member to the second region; and
a step of removing the solution containing the intestinal flora, accommodated in the second region, to the outside of the vessel main body.

17. The intestinal flora extraction method according to claim 16,
wherein in the step of accommodating the solution, a syringe, a bag, or a bottle is connected to the vessel main body, and the solution is injected from the syringe, the bag, or the bottle into the vessel main body, and
in the step of removing the solution containing the intestinal flora to the outside of the vessel main body, the solution containing the intestinal flora is discharged into the syringe, the bag, or the bottle, which remains connected to the vessel main body.

18. The intestinal flora extraction method according to claim 16, wherein the solution is at least one of physiological saline, Ringer's solution, Ringer's lactate solution, or a solution to which a component with an excellent ability to support the growth of intestinal flora has been added.

19. The intestinal flora extraction method according to claim 18, wherein the component with an excellent ability to support the growth of intestinal flora is at least one of an amino acid, a sugar, a mineral, a vitamin, and lactic acid bacteria, or the component is a yogurt containing at least one of these.

20. The intestinal flora extraction method according to claim 16, further comprising:
a step of attaching the prepared vessel main body to a toilet such that the first region side opens,
wherein in the step of accommodating the feces into the filter member, the feces discharged by a subject person sitting on the toilet is directly accommodated into the filter member.

21. The intestinal flora extraction method according to claim 16, further comprising:
a step of covering the vessel main body with an outer vessel,
wherein the application of external force to the vessel main body is performed from the outside of the outer vessel.

22. The intestinal flora extraction method according to claim 21, wherein at least a part of the outer vessel is non-transparent or colored.
